(19) United States
(12) Patent Application Publication
(10) Pub. No.: US 2001/0033869 A1
(43) Pub. Date: Oct. 25, 2001

(54) Utilization of an egg-based preparation as an antidepressant

(75) Inventor: Bjodne Eskeland, Oslo (NO)

Correspondence Address: YOUNG & THOMPSON, 745 SOUTH 23RD STREET 2ND FLOOR, ARLINGTON, VA, 22202

(73) Assignee: Drymed AS

(21) Appl. No.: 09/865,443

(22) Filed: May 29, 2001

Related U.S. Application Data
(63) Continuation of application No. 09/308,389, filed on May 18, 1999, now abandoned.

(30) Foreign Application Priority Data
Nov. 20, 1996 (NO) 19964935

(51) Int. Cl. A61K 35/54

(52) U.S. Cl. 424/520

(57) ABSTRACT

The utilization of an egg-based preparation as an antidepressant.

The present invention relates to the use of an egg-based preparation as an antidepressant.

More specifically, the invention relates to the utilization of a preparation corresponding to the one described in NO 922988 and the WO 94/03192 based thereon, as an antidepressant.

In NO application 922988 there is described a potency enhancing agent based on a powder produced from fertilized avian eggs.

The application is directed particularly toward a powder of eggs obtained 10 days after fertilization.

In accordance with NO application 962886, the egg preparation corresponding to the ones in NO 922988 also seemed to have a muscle building and strength enhancing effect when used.

Further investigations seem to suggest that preparations of this type also have an antidepressant effect.

Preliminary studies carried out on persons for whom antidepressant medications had not been prescribed were examined over an 8-week period, with results which were clearly indicative of an antidepressant effect.

A study was thus carried out on 6 mentally depressed persons.

These subjects were administered the product in an amount of 3 g×2 per day for 8 weeks.

The product is based on avian eggs which have been fertilized and incubated for about 10 days.

The three criteria which the subjects were asked to evaluate were:

1) self-esteem,

2) well-being (“happiness”), and

3) energy level.

The individuals were asked to evaluate the three factors of self-esteem, well-being and energy level each week for 8 weeks, utilizing a visual analogue scale ranging from 0 cm=no change, to 10 cm=very pronounced change, said scale having a midpoint of 5 representing the normal condition.

The results are summarized in Table 1.

TABLE 1
Changes in self-esteem, well-being and energy level before and
after an 8-week treatment.

| | Before | After 8 weeks |
|---|---|---|
| Self-esteem | 1.8 +/− 0.3 | 6.4 +/− 0.8 |
| Well-being | 1.1 +/− 0.3 | 5.9 +/− 0.7 |
| Energy level | 2.8 +/− 0.7 | 4.6 +/− 1.0 |

In the course of the 8-week periods, the participants in this study consumed the recommended daily dosage; and, as is indicated in the table, the level of self-esteem increased by a factor of 3.5, well-being increased by a factor of 5.4 and energy level rose by a factor of 1.6.

The weekly change was examined, and these results are given in Table 2.

TABLE 2
Response on a weekly basis for 8 weeks when 6 individuals
were given the product.

| | Weeks of treatment | | | | | | |
|---|---|---|---|---|---|---|---|
| | 0 | 1 | 2 | 3 | 4 | 6 | 8 |
| Self-esteem | 1.8 | 1.8 | 2.8 | 3.1 | 4.0 | 5.1 | 5.9 |
| Well-being | 1.1 | 1.6 | 2.6 | 3.9 | 4.1 | 5.0 | 5.9 |
| Energy level | 2.8 | 2.9 | 3.4 | 4.4 | 4.4 | 4.6 | 4.6 |

For the three criteria measured, it is apparent from Table 2 that a gradual increase occurred after the first week and throughout the entire test period of 8 weeks.

The values reported above are the average of what was obtained from the 6 subjects. The conclusion from this, based on the judgments for 1 to 3 above, is that the evaluated product clearly seems to have a significant antidepressant effect.

The product may be consumed as is, but it may also advantageously be ingested with the aid of one or more physiologically acceptable carriers or fillers.

What is claimed is:

1. A method for treatment of mental depression, comprising administering to a patient in need of said treatment an effective amount of a composition comprising dried fertilized chicken eggs that have been incubated about 10 days.

2. The method of claim 1, wherein said composition is administered twice daily for at least about 4 weeks.

3. The method of claim 1, wherein said composition is administered twice daily for at least about 8 weeks.
